(19) United States
(12) Patent Application Publication
(10) Pub. No.: US 2024/0066056 A1
(43) Pub. Date: Feb. 29, 2024

(54) Formulations of a Prophylactic Nasal Spray and Methods of Use and Manufacture Thereof

(71) Applicant: NTBY Moss LLC, San Antonio, TX (US)

(72) Inventor: Jesse Moss, Jr., San Antonio, TX (US)

(73) Assignee: NTBY Moss LLC, San Antonio, TX (US)

(21) Appl. No.: 18/260,222

(22) PCT Filed: Jan. 3, 2022
(86) PCT No.: PCT/US22/70005

§ 371 Date: Jun. 30, 2023

Related U.S. Application Data
(60) Provisional application No. 63/132,608, filed on Dec. 31, 2020.

(51) Int. Cl. A61K 33/18; A61K 9/00; A61K 9/08

(52) U.S. Cl. CPC A61K 33/18; A61K 9/0043; A61K 9/08

(57) ABSTRACT

Methods, formulations, and uses of a prophylactic nasal spray are provided. Many embodiments include nasal spray formulations including an microbicidal (e.g., virucidal) agent. Various embodiments include iodine, such as povidone iodine (PVP-I) as the microbicidal agent. Additional embodiments include methods to prophylactically treat an individual to prevent, mitigate, or otherwise limit a microbial infection.

### CROSS-REFERENCE TO RELATED APPLICATIONS

The current application claims priority to U.S. Provisional Patent Application No. 63/132,608, entitled “Airborne Viral Prophylactic Nasal Spray Containing Iodine” by Jesse Moss Jr., filed Dec. 31, 2020; the disclosure of which is hereby incorporated by reference in its entirety.

### FIELD OF THE DISCLOSURE

The present disclosure relates to formulations of a prophylactic nasal spray, including methods of use and manufacture of the same. More specifically, the present disclosure relates to nasal sprays including a microbicidal and/or virucidal agent to prevent, mitigate, or otherwise limit a microbial infection.

### BACKGROUND OF THE DISCLOSURE

Severe acute respiratory syndrome coronavirus 2 (SARS-CoV-2), the virus resulting in coronavirus disease 2019 (COVID-19), is a novel coronavirus in the same family as severe acute respiratory syndrome coronavirus (SARS-CoV) and Middle East respiratory syndrome (MERS). On Mar. 11, 2020, this rapidly spreading novel coronavirus, SARS-CoV-2 (COVID-19) was declared a pandemic by the World Health Organization.

Per the World Health Organization, the SARS-CoV-2 virus spreads between people, mainly when an infected person is in close contact with another person. The virus can spread from an infected person's mouth or nose in small liquid particles when they cough, sneeze, speak, sing, or breathe heavily. These liquid particles are different sizes, ranging from larger “respiratory droplets” to smaller “aerosols.”

People can catch COVID-19 when the virus gets into their mouth, nose, or eyes, which is more likely to happen when people are in direct or close contact (less than 1 meter apart) with an infected person. Current evidence suggests that the main way the virus spreads is by respiratory droplets among people who are in close contact with each other.

Aerosol transmission can occur in specific settings, particularly in indoor, crowded and inadequately ventilated spaces, where infected person(s) spend long periods of time with others. (See e.g., World Health Organization, “Coronavirus disease (COVID-19): How is it transmitted?”, updated Oct. 20, 2020, page 1, www.who.int/news-room/q-a-detail/coronavirus-disease-covid-19-how-is-it-transmitted; the disclosure of which is hereby incorporated by reference in its entirety.) As such, there is a need in the art for devices, solutions, and/or methods that can mitigate or limit the ability of a virus, bacteria, or other microbe to infect someone.

### SUMMARY OF THE DISCLOSURE

This summary is meant to provide examples and is not intended to be limiting of the scope of the invention in any way. For example, any feature included in an example of this summary is not required by the claims, unless the claims explicitly recite the feature. Also, the features described can be combined in a variety of ways. Various features and steps as described elsewhere in this disclosure can be included in the examples summarized here.

In one embodiment, a prophylactic nasal spray includes an aqueous solution including an microbicidal agent at an effective concentration to limit a microbial infection.

In a further embodiment, the microbicidal agent is selected from one or more of povidone iodine (PVP-I), elemental iodine, molecular iodine, and an iodine salt.

In another embodiment, the iodine concentration is at least 0.0001%.

In a still further embodiment, the microbicidal agent is povidone iodine (PVP-I) is at a concentration of approximately 0.1% to approximately 25%.

In still another embodiment, the microbicidal agent is povidone iodine (PVP-I) is at a concentration of approximately 2.3%.

In a yet further embodiment, the aqueous solution is a hypertonic solution.

In yet another embodiment, the aqueous solution includes a salt selected from sodium chloride, potassium chloride, sodium phosphate, sodium bisphosphate, and sodium acetate.

In a further embodiment again, the aqueous solution further includes a buffer.

In another embodiment again, the buffer is sodium bicarbonate.

In a further additional embodiment, the prophylactic nasal spray further includes a delivery container, where the delivery container is capable of distributing the nasal spray into a nasal cavity of an individual.

In another additional embodiment, the delivery container is an Aptar classic or equivalent sprayer.

In a still yet further embodiment, the aqueous solution has a pH of 6.3±0.6.

In still yet another embodiment, a method of prophylactically treating an individual against a microbial infection including obtaining a nasal spray comprising an aqueous solution including an microbicidal agent at an effective concentration to limit a microbial infection and delivering the nasal spray to a nasal cavity of an individual.

In a still further embodiment again, the method further includes cleaning the nasal cavity of the individual.

In still another embodiment again, cleaning the nasal cavity includes blowing the nose of the individual or flushing the nasal cavity of the individual with a fluid.

In a still further additional embodiment, cleaning the nasal cavity further includes introducing a decongestant to the nasal cavity of the individual.

In still another additional embodiment, the microbicidal agent is povidone iodine (PVP-I) is at a concentration of approximately 0.1% to approximately 25%.

In a yet further embodiment again, the microbicidal agent is povidone iodine (PVP-I) is at a concentration of approximately 2.3%.

In yet another embodiment again, the aqueous solution is a hypertonic solution.

In a yet further additional embodiment, the aqueous solution further includes a buffer.

In yet another additional embodiment, the nasal spray is obtained in a delivery container capable of distributing the nasal spray into a nasal cavity of an individual.

In a further additional embodiment again, the aqueous solution has a pH of 6.3±0.6.

In various embodiments, the methods can be performed on a living animal (including a human) or on a non-living cadaver, cadaver head, simulator (e.g. with the body parts, tissue, etc. being simulated), anthropomorphic ghost, etc.

The foregoing and other objects, features, and advantages of the disclosed technology will become more apparent from the following detailed description, which proceeds with reference to the accompanying figures.

### BRIEF DESCRIPTION OF THE DRAWINGS

FIG. 1 illustrates an exemplary sagittal view of an individual in accordance with various embodiments of the invention.

FIG. 2 illustrates a box diagram illustrating a method of prophylactically treating an individual against a microbial infection in accordance with various embodiments of the invention.

### DETAILED DESCRIPTION OF THE DISCLOSURE

Turning now to the drawings and data, embodiments of the invention are generally directed to formulations a prophylactic nasal spray, including methods of use and manufacture thereof. Many embodiments include iodine within the nasal spray for antiviral, antibacterial, antimicrobial, virucidal, bactericidal, and/or microbicidal activity or properties. Certain embodiments deliver the nasal spray into the nasal cavity of an individual. Many embodiments are prophylactic against viruses, bacteria, and other microbes.

A mucosa, or mucous membrane, is a membrane that lines various cavities in the body and covers the surface of internal organs. It consists of one or more layers of epithelial cells overlying a layer of loose connective tissue. Mucosa exist in several places of the human body, including the nasal cavity, various sinus cavities. Additional non-limiting examples mucosa include the bronchial mucosa, endometrium, esophageal mucosa, gastric mucosa, intestinal mucosa, oral mucosa, penile mucosa, vaginal mucosa, frenulum, the tongue, the anal cavity, palpebral conjunctiva, and many others.

Many infections, such as caused by bacterial, viral, fungal, and other microbes, enter the body via mucosa, including respiratory infections, such as COVID-19. Studies have shown that in symptomatic COVID-19 patients, nasal swabs have yielded higher viral loads than throat swabs. The same distribution was observed in an asymptomatic patient, implicating the nasal epithelium as a portal for initial infection and transmission.

Cellular entry of coronaviruses depends on the binding of the spike (S) protein to a specific cellular receptor and subsequent S protein priming by cellular proteases. Additionally, SARS-CoV-2 employs angiotensin-converting enzyme 2 (ACE2) as a receptor for cellular entry. In SARS-CoV, the binding affinity of the S protein and ACE2 was found to be a major determinant replication rate and disease severity. Scientists have studied how goblet and ciliated cells in the nose have high levels of the entry proteins that airborne viruses like COVID-19 use to get into our cells, thus the specific nose cell types have been studied to show that they are likely initial infection points for airborne viral infections like COVID-19. (See e.g., Sungnak, W., Huang, N., Bécavin, C. et al. SARS-CoV-2 entry factors are highly expressed in nasal epithelial cells together with innate immune genes. Nat Med 26, 681-687 (2020); the disclosure of which is hereby incorporated by reference in its entirety.)

Given the potential for airborne infection via the nasal mucosa, besides masks, additional respiratory hygiene measures can be considered to reduce the risk of airborne virus transmission. Removing or killing a virus after it settles on the mucous membranes of the eyes, nose, or mouth but before it infects the cell offers another option for reducing systemic infection to the virus. Nasal antiseptics and nasal rinses as well as throat gargles and mouthwashes have shown in vitro effectiveness against these airborne infections.

Povidone-iodine (PVP-I), also known as iodopovidone, is an antiseptic that has been in commercial use since 1955. It is a chemical complex of povidone, hydrogen iodide and elemental iodine. It exhibits longer lasting antiseptic effects than a tincture of iodine, which is typically 2-7% elemental iodine dissolved in a mixture of ethanol and water. PVP-I is soluble in cold and mild-warm water and alcohol, and in a proper dilute solution, it is a broad-spectrum antiseptic with powerful microbicidal properties, including bactericidal and virucidal activity.

PVP-I formulations have been widely used for over 60 years because of an established safety profile, and PVP-I is also listed on the World Health Organizations' List of Essential Medicines. The uses of PVP-I have commonly been used in medical fields as a topical antiseptic application prior to and after a surgery or other invasive procedure to disinfect the area of interest and prevent infection. An application of povidone-iodine has been shown to have bactericidal action for up to eight hours after application. (See e.g., Timothy J. King, Edward M. Sullivan, in Physician Assistant (Fourth Edition), 2008; the disclosure of which is hereby incorporated by reference in its entirety.)

PVP-I has also been used in both intranasal preparations against Methicillin Resistant Staphylococcus Aureus (MRSA) as well as oral preparations in in vitro studies of Severe Acute Respiratory Syndrome Coronavirus (SARS-CoV), Middle Eastern Respiratory Syndrome Coronavirus (MERS-CoV), influenza H1N1, and rotavirus with good efficacy. Specifically, to SARS-CoV-2, PVP-I has demonstrated rapid in vitro virucidal activity, killing 99.99% of the virus within 30 seconds of contact. (See e.g., Anderson, D. E., Sivalingam, V., Kang, A. E. Z. et al. Povidone-Iodine Demonstrates Rapid In Vitro Virucidal Activity Against SARS-CoV-2, The Virus Causing COVID-19 Disease. Infect Dis Ther 9, 669-675 (2020); the disclosure of which is hereby incorporated by reference in its entirety.)

While the World Health Organization recommends the use of personal protection equipment, including masks, additional prophylactic approaches will provide additional prevention opportunities. As such, many embodiments comprise a nasal spray including iodine, such as PVP-I, as a preventative for respiratory infections. While there are examples of nasal antiseptics commercially available, many of these products are alcohol-based or contain other primary active, which can be harmful to the nasal cavity if used regularly. There are examples of iodine-based antiseptics in treatment of chronic rhinosinusitis and nasal congestion, but these products are swabs or topical applications, which cannot reach the mucosa, thus cannot prevent infection and/or cleanse areas where microbes can take hold.

### Nasal Sprays

Many embodiments provide a nasal spray with an antimicrobial (e.g., antiviral) or microbicidal (e.g., virucidal) agent. In certain embodiments, the nasal spray is an aqueous solution, and various embodiments include iodine as the antimicrobial or microbicidal agent. Many of these embodiments act as a prophylactic against airborne microbial infections, including viral infections (such as SARS-CoV-2), bacterial infections, fungal infections, and other microbial infections. The formulation, including iodine, of many embodiments provides reversible toxicity to the cilia of the nasal mucosa with a fast onset of action and for a prolonged period of time as a method of preventing SARS-CoV-2 and other viruses from entering the cell of the nasal mucosa.

In some embodiments, the iodine is selected from one or more of molecular iodine, elemental iodine, ionic iodine (including hydrogen iodide and other iodine salts), and complexed iodine (including polymers with bound iodine). Certain embodiments include povidone iodine (PVP-I) as the iodine within the nasal spray, while other embodiments molecular iodine. In many embodiments, the iodine concentration has an effective concentration that is sufficient to provide reversible toxicity to the cilia of nasal mucosa. The reversible toxicity stops transport of viruses, bacteria, fungus, or other microbes into a cell.

Various include PVP-I at a concentration (w/w) of approximately 0.1%, 0.5%, 1.0%, 2.5%, 5%, 10%, 15%, 20%, 25%, or more. PVP-I is approximately is approximately 10% (e.g., ±2%) of available iodine, thus the above concentrations of PVP-I would have a concentration of available iodine is 1/10 of the PVP-I concentration. For example, in an embodiment that is 10% PVP-I, the available iodine would have a concentration of 1%, while an embodiment that is 5% PVP-I, the available iodine would have a concentration of 0.5%. Some preferred embodiments include a PVP-I concentration from 5-10% (thus 0.5%-1% available iodine). However, certain embodiments dilute a 10% (w/w) PVP-I solution to be 15%-30% (v/v) of the total volume of the nasal spray. In some embodiments using molecular iodine, the iodine concentration is at least 0.00001% (w/w). As such, some of these embodiments have an iodine concentration of approximately 0.0001%, 0.0001%, 0.001%, 0.01%, 0.05%, 0.1%, 0.5%, 1%, or more, with a preferred range of approximately 0.0001%-0.05% (w/w) of molecular iodine.

Further embodiments include solutes, including ionic solutes to create a hypertonic solution. Examples of molecules included in various embodiments include sodium chloride, potassium chloride, sodium phosphate, sodium bisphosphate, and sodium acetate. In many embodiments, the solutes are at a concentration so the nasal spray is hypertonic in relation to the mucosa. The hypertonic solution of certain embodiments may draw out fluid and mucous from the mucosa. In many embodiments, the solution is at least 2% (w/v) salt or other solute, and many embodiments use one or more of sodium chloride and sodium phosphate to achieve the hypertonic concentration. In some embodiments, the solute concentration is 2%, 2.5%, 3%, 3.5%, 4%, 4.5%, or 5% (w/v).

Various embodiments include one or more buffers, preservatives, and other molecules to aid in efficacy, stability, and/or any other property of the nasal spray. Buffers, in accordance with some embodiments, can lessen irritation of mucosa or other tissue caused by iodine, salts, and/or other compounds within the nasal spray. In many embodiments, the buffer is a biocompatible buffer—e.g., a buffer that can be broken down by a body and/or does not cause any irritation to a body, such as sodium bicarbonate, potassium bicarbonate, sodium citrate, and/or other buffers that can be used for medical, pharmaceutical, and/or cosmetic purposes. In certain embodiments, the buffer is sodium bicarbonate. Various provide a buffer in a concentration to have a pH similar to the average nasal pH (e.g., approximately 6.3±0.6), while certain embodiments buffer the nasal spray to a neutral pH (e.g., pH 7±0.5).

Certain embodiments manufacture the nasal spray by diluting a PVP-I solution with a hypertonic solution. In such embodiments, a PVP-I solution is obtained from a source (e.g., commercial manufacturer), and the hypertonic solution is manufactured or mixed, such that the PVP-I and hypertonic solution dilute each other to a final concentration. In some embodiments, the hypertonic solution includes one or more buffers, preservatives, or other compounds for stability, salinity, or other property. In some embodiments, the hypertonic solution is approximately 3.5% (w/v) (e.g., ±0.5%) saline (sodium chloride). In additional embodiments, the hypertonic includes a buffering agent. Some of these embodiments use sodium bicarbonate as the buffer at a concentration of approximately 0.35% (w/v) (e.g., ±0.15%) of the buffering agent.

An exemplary embodiment of the solution is provided in the following table:

Based on 1 L:

| | |
|---|---|
| 23% | 10% PVP-I |
| 77% | Hypertonic Saline Solution |
| | Water |
| | 3.6 g sodium bicarbonate |
| | 34.12 g sodium chloride |

Mixing the above solutions yields a final solution of approximately 0.28% (w/v) sodium bicarbonate, approximately 2.63% (w/v) sodium chloride, and approximately 2.3% (v/v) PVP-I.

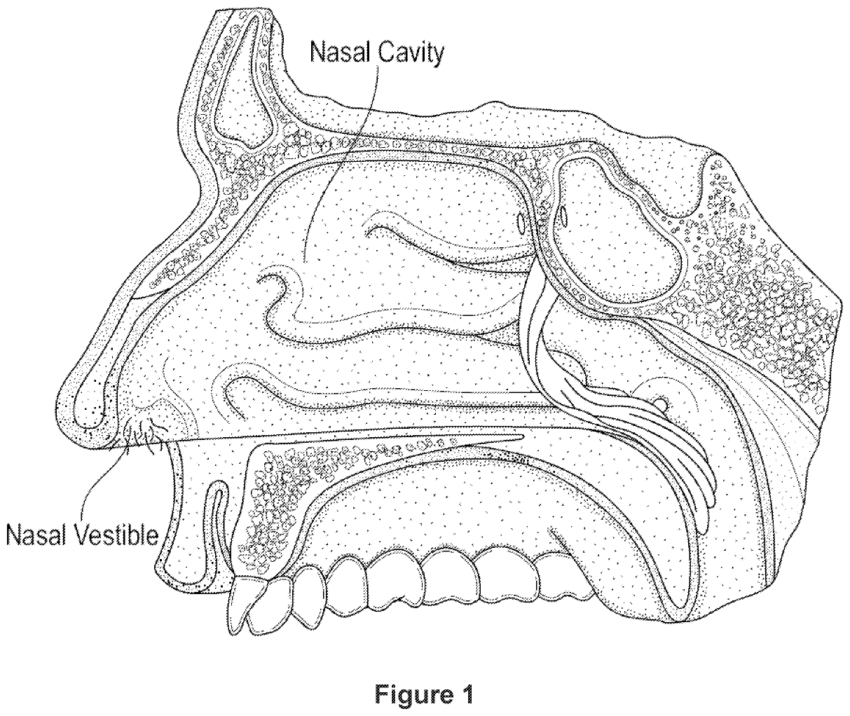

Various embodiments provide the nasal spray in an applicator that is capable of delivering the nasal spray solution to the nasal cavity. FIG. 1 illustrates a sagittal view of a human head, emphasizing a nasal cavity. As illustrated in FIG. 1, the nasal cavity is connected to a nasal vestibule, which allows access to the nasal cavity from external devices. On average, the nasal cavity possesses approximately 160 cm² of surface area—or 96 m², if the surface area of microvilli is included. In various embodiments, the applicator is a spray bottle. In many such embodiments, the spray bottle is capable of nebulizing or aerosolizing the nasal spray. Many embodiments utilize sprayer capable of aerosolizing or nebulizing the spray to get beyond the nasal vestibule and into the nasal cavity. Certain embodiments provide the nasal spray as a cloud or nebula immediately from the applicator/spray-bottle, while some embodiments provide the nasal spray as a stream that breaks into a cloud or nebula after a certain distance. In some of these embodiments, the distance of the stream is sufficient to reach the nasal cavity before aerosolizing or nebulizing the nasal spray.

As such, In many embodiments, the sprayer delivers the aerosolized or nebulized spray in a dose sufficient to coat the mucosa within the nasal cavity. Further embodiments nebulize the dose to a sufficient rate, such that the droplets are able to reach most of the mucosa, such as greater than 50% of the mucosa, greater than 60% of the mucosa, greater than 70% of the mucosa, greater than 75% of the mucosa, greater than 80% of the mucosa, greater than 85% of the mucosa, greater than 90% of the mucosa, or greater than 95% of the mucosa. Some embodiments are capable of coating substantially all (e.g., 95-100%) of the mucosa. Certain embodiments provide a dose of approximately 70 μL of the nasal spray into the nasal cavity. However, additional embodiments provide smaller doses for children (e.g., approximately 25-50 μL) depending on age or size of the individual, while some embodiments provide a larger dose such as approximately 90-95 μL for individuals who may have an enlarged nasal cavity. As noted previously, the aerosolized or nebulized spray is delivered to the nasal cavity. As such, some embodiments may provide a slightly higher volume in a dose of nasal spray, such that the volume that enters the nasal cavity is approximately 70 μL—or any other dose for an individual, such as less for children and/or sensitive individuals or more for individuals with enlarged nasal cavities. Some embodiments utilize an Aptar classic or equivalent sprayer to deliver a nasal spray.

### Methods of Providing a Nasal Spray to an Individual

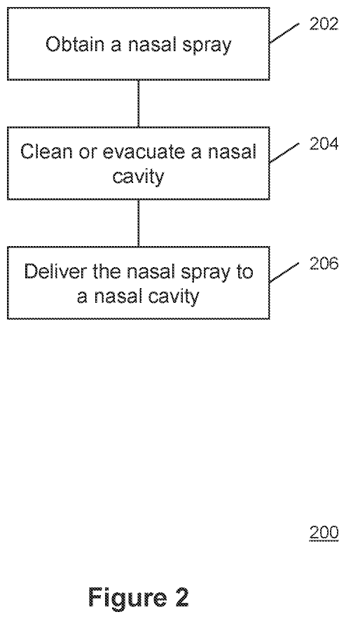

Turning to FIG. 2, various embodiments are directed to a method 200 of delivering a nasal spray to an individual. As described herein, embodiments of nasal sprays can be used as a prophylactic against microbial infection, including infections caused by viruses, bacteria, fungi, and other microbes. Some of these embodiments utilize iodine for its virucidal and other microbicidal properties.

At 202 of many embodiments, obtain a nasal spray. In various embodiments, the nasal spray is obtained in a spray bottle, sprayer, or other delivery container capable of distributing the nasal spray into a nasal cavity of an individual. However, certain embodiments obtain the nasal spray as a bulk solution, then portion, aliquot, or otherwise divide the bulk solution into a spray bottle, sprayer, or other delivery container. Further embodiments obtain the nasal spray by manufacturing or mixing the solution in accordance with the embodiments described here, then portion, aliquot, or otherwise divide the solution into a spray bottle, sprayer, or other delivery container.

Some embodiments clean or evacuate the nasal cavity of an individual at 204 to remove excess mucus and/or debris from the nasal cavity. In certain embodiments, the cleansing of the nasal cavity is accomplished by the individual blowing their nose. Various embodiments flush the nasal cavity with a solution or fluid, such as water or saline solution, including by using a device such as a Neti Pot, Navage, or other device or system that can flush an individual's nasal cavity. Some embodiments introduce a decongestant to clear mucus, congestion, or other natural obstruction from the nasal cavity.

Additional embodiments deliver the nasal spray to the nasal cavity at 206. In such embodiments, a delivery container (e.g., sprayer, spray bottle, etc.) is positioned in the nasal vestibule (e.g., nostril) of an individual, such that the exit point (e.g., pore) on the delivery container is positioned within the nasal vestibule. Many embodiments further actuate the delivery container to disperse the nasal spray into the nasal cavity. In some embodiments, the individual inhales through the nose simultaneously with actuation of the delivery container to help distribute the nasal spray within the nasal cavity. In many embodiments, delivering the nasal spray is repeated for the second nasal vestibule—e.g., the delivery container is positioned at the other nasal vestibule and actuated to disperse the nasal spray into the nasal cavity.

It should be noted various embodiments may repeat, omit, or change the order of certain steps within the scope of this disclosure. For example, cleaning or evacuating the nasal cavity 204 may be omitted if there is no concern for natural obstructions. An additional example repeats some or of method 200 on various intervals to maintain the presence of the nasal spray throughout a day, week, month, or season (including natural seasons such as Autumn and Winter or colloquial seasons such as Flu Season or Awards Season). In such embodiments, intervals can include every 30 minutes, 60 minutes, 120 minutes, 3 hours, 4 hours, 6 hours, 8 hours, 12 hours, 24 hours, etc., such that the iodine is present within the nasal cavity at an effective concentration for preventing, mitigating, or otherwise limiting a microbial (e.g., viral) infection.

### Doctrine of Equivalents

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the foregoing examples and descriptions of various preferred embodiments of the present invention are merely illustrative of the invention as a whole, and that variations in the components or steps of the present invention may be made within the spirit and scope of the invention. Accordingly, the present invention is not limited to the specific embodiments described herein, but, rather, is defined by the scope of the appended claims.

1. A prophylactic nasal spray comprising:
an aqueous solution including an microbicidal agent at an effective concentration to limit a microbial infection.

2. The prophylactic nasal spray of claim 1, wherein the microbicidal agent is selected from one or more of povidone iodine (PVP-I), elemental iodine, molecular iodine, and an iodine salt.

3. The prophylactic nasal spray of claim 2, wherein the iodine concentration is at least 0.0001%.

4. The prophylactic nasal spray of claim 1, wherein the microbicidal agent is povidone iodine (PVP-I) is at a concentration of approximately 0.1% to approximately 25%.

5. The prophylactic nasal spray of claim 1, wherein the microbicidal agent is povidone iodine (PVP-I) is at a concentration of approximately 2.3%.

6. The prophylactic nasal spray of claim 1, wherein the aqueous solution is a hypertonic solution.

7. The prophylactic nasal spray of claim 1, wherein the aqueous solution includes a salt selected from sodium chloride, potassium chloride, sodium phosphate, sodium bisphosphate, and sodium acetate.

8. The prophylactic nasal spray of claim 1, wherein the aqueous solution further includes a buffer.

9. The prophylactic nasal spray of claim 8, wherein the buffer is sodium bicarbonate.

10. The prophylactic nasal spray of claim 1, further comprising a delivery container, wherein the delivery container is capable of distributing the nasal spray into a nasal cavity of an individual.

11. The prophylactic nasal spray of claim 10, wherein the delivery container is an Aptar classic or equivalent sprayer.

12. The prophylactic nasal spray of claim 1, wherein the aqueous solution has a pH of 6.3±0.6.

13. A method of prophylactically treating an individual against a microbial infection, comprising:
obtaining a nasal spray comprising an aqueous solution including an microbicidal agent at an effective concentration to limit a microbial infection; and
delivering the nasal spray to a nasal cavity of an individual.

14. The method of claim 13, further comprising cleaning the nasal cavity of the individual.

15. The method of claim 14, wherein cleaning the nasal cavity comprises blowing the nose of the individual or flushing the nasal cavity of the individual with a fluid.

16. The method of claim 14, wherein cleaning the nasal cavity further comprises introducing a decongestant to the nasal cavity of the individual.

17. The method of claim 13, wherein the microbicidal agent is povidone iodine (PVP-I) is at a concentration of approximately 0.1% to approximately 25%.

18. The method of claim 13, wherein the microbicidal agent is povidone iodine (PVP-I) is at a concentration of approximately 2.3%.

19. The method of claim 13, wherein the aqueous solution is a hypertonic solution.

20. The method of claim 13, wherein the aqueous solution further includes a buffer.

21. The method of claim 13, wherein the nasal spray is obtained in a delivery container capable of distributing the nasal spray into a nasal cavity of an individual.

22. The method of claim 13, wherein the aqueous solution has a pH of 6.3±0.6.
